(19) United States
(12) Patent Application Publication
(10) Pub. No.: US 2024/0221916 A1
(43) Pub. Date: Jul. 4, 2024

(54) DocMoonlight: System and Method for In-training Medical Personnel Recruitment

(71) Applicant: Ramez Morcos, Lake Worth, FL (US)

(72) Inventor: Ramez Morcos, Lake Worth, FL (US)

(21) Appl. No.: 18/093,270

(22) Filed: Jan. 4, 2023

(51) Int. Cl. G16H 40/20; G06Q 10/1053

(52) U.S. Cl. CPC G16H 40/20; G06Q 10/1053

(57) ABSTRACT

This disclosure generally relates to a novel program that aims to provide work opportunities for in-training healthcare staff and allows them to register at the platform wherein the HCSP position (s) will be matched to available ITMS(s) per their pre-set requirements. The system further provides multiple exemplary features to ensure ease of use, consistency and reliability to both parties.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

This invention relates generally to methods and systems for staffing of healthcare staff and more particularly relates to a system and method to connect trainee doctors, nurses, technicians, etc. [i.e., referred to as in-training medical staff (ITMS)] to healthcare service providers who need temporary staff [i.e., referred to as Health Care Service Providers (HCSP)] with the aim to match ITMS with HCSP more efficiently.

### DESCRIPTION OF THE RELATED ART

There is a niche market for filling short-term or temporary medical staff positions. Very few medical staffing companies exist in this market because of the fast response needed and the high overhead incurred to perform such activities. ITMS are busy with their ongoing training to search on their own for moonlighting opportunities as the positions are temporary. This causes a problem because it often limits ITMS ability to find and interact with HCSP during business hours only. HCSPs do not have the workforce to try to find ITMS to fill these temporary medical positions themselves. Therefore, one limiting factor is the cost of having someone available to call each ITMS and determine their eligibility and interest in the position. Staffing of temporary medical positions may include staffing a position for a few hours or a few days based on sudden demands. In many cases, the potential ITMs will need to meet with HCSP to explore the moonlighting opportunities, which means that they will have to travel, which costs a great deal in terms of travel expenses, including transportation, lodging, food, and other essential travel expenses without a guarantee that the position is suitable to their need. This is a fundamental problem in the short-term medical position staffing industry.

By looking at prior art, in response to staffing needs and available staff, the proposed system is a solution that matches ITMS with HCSP for a temporary position. The current invention proposes an advanced methodology of presenting an onboard registration, application, background check, and verification that allows ITMS to be connected to HCSP service providers with a need for short-term positions. As a result, the system saves time, money, resources, and talent while being more effective for all parties involved.

Thus it has been noted, to Applicant's knowledge, none of these prior art methods is entirely suitable to meet these needs and would be cumbersome in trying to alleviate the current needs. Therefore, there is a need in the prior art in which the inconveniences, as mentioned earlier, difficulties, and grooming problems are eliminated for all practical purposes. Thus, the present invention provides such a method, and the overall combination of these features is nowhere disclosed in the prior art cited above, which appears to represent the general art in this area, although it is not intended to be an all-inclusive listing of pertinent prior art patents.

### SUMMARY

In light of the disadvantages of the prior art, the following summary is provided to facilitate an understanding of some of the innovative features unique to the present invention and is not intended to be a complete description. A full appreciation of the various aspects of the invention can be gained by taking the entire specification, claims, drawings, and abstract as a whole.

The present subject matter relates generally to an improved system related to connecting ITMS to HCSP.

It is also the objective of the system to provide software that includes a profile database, which stores information about the ITMS and HCSP. The information stored about the ITMS includes availability, services performed, geographic service area, experience, etc . . . .

Another embodiment of the invention is that aspect, through an online system where the system involves, a service request message is received from a HCSP via a first communication device or a longer-term work calendar is established. The service request message includes a description of a requested service, a date and time period for the performance of the requested service and a location where the requested service is to be performed. The preferences of the ITMS are retrieved from the profile database and matched to the job specification is is created from the service request message.

Another objective of the system is to provide an online application that triggers the in-house management system to process, verify and communicate with the Accreditation Council for Graduate Medical Education (ACGME) training programs to ensure that the doctor-in-training is limited to duty hours (80 hours/week). Furthermore, the in-house system will verify and provide background checks for any and all ITMS staff operating within the system.

It is accordingly an object of the invention to provide a dedicated web and client application that is empowered through a graphical user interface (GUI) consumer-based interface, while the feedback is stored and sorted through a database server at the backend.

Another objective of the system is to provide a system that in particular, provides two business models. First, a revenue stream based on a commission fee that is earned from the ITMS workers (i.e., a percentage for each shift worked). The total value of each shift includes the doctor-in-training payment, fees, taxes, and company commission. Second, a subscription fee is obtained from the HCSP to use the automated features of the platform and to maintain constant coverage for shifts.

It is also the objective of the invention to contract with multiple HCSP that need ITMS to work and obtain an additional source of income with flexible working schedules. In addition, HCSP will deliver high-quality patient care by utilizing the ITMS talents and decreasing the high pay rate of senior staff.

It is moreover the objective of the invention to provide two revenue streams. One is to act as an intermediary party where the service provider will bill the companies with total hours per location and then disburse the payments among health care staff.

It is also the objective of the invention to provide secondary revenue stream subscription or one-time fee per location or monthly fee, when HCSP access the platform features and integrate the efficient communication between HR, site manager, and ITMS,

Although the invention is illustrated and described herein as embodied in an online ITMS recruitment system, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

Other objects, features and advantages of the present invention will be apparent from the accompanying drawing and from the detailed description which follows.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 shows working views of proposed invention as per preferred embodiments of the invention.

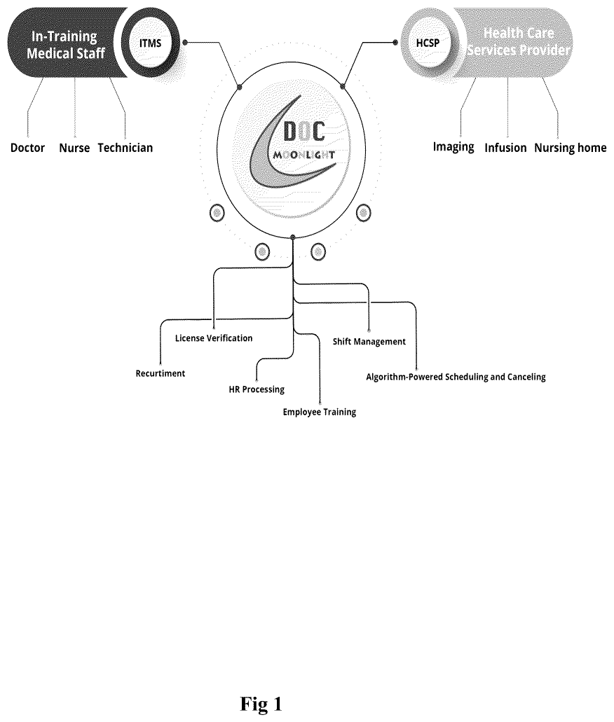

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The present invention now will be described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as methods or devices. Accordingly, the present invention may take the form of an entirely hardware embodiment, an entirely software embodiment or an embodiment combining software and hardware aspects. The following detailed description is, therefore, not to be taken in a limiting sense.

The current invention as per its preferred embodiment provides a method allowing ITMS to find contract-level jobs, whereas HCSP can find doctors, nurses, technicians, etc. as per their requirements through this application.

The invention is based on components of an exemplary environment in which the invention may be used. The online system has focused components and is based on simple-to-use technology. The software can be viewed and used from personal computers, multiprocessor systems, microprocessor-based or programmable consumer electronics, network PCs, and the like. In order to use the application, the user (i.e., user could be ITMS or HCSP) has to be connected to the internet to use the services in real-time. The real-time utility needs to be connected to the internet through local area networks (“LANs”)/wide area networks (“WANs”), wireless networks, or independent internet sources. Further, where applicable, the internet would connect the user to the online application server, through the user's computing or mobile device.

Generally, the client (i.e., client could be ITMS or HCSP) device may include virtually any computing device capable of receiving and sending a message over a network, such as LAN/WAN or independent network, wireless network, and the like, to and from another computing device, such as a server or mobile device, and the like. The set of such devices may include devices that typically connect using a wired communications medium such as personal computers, multiprocessor systems, microprocessor-based or programmable consumer electronics, network PCs, and the like. The set of such devices may also include devices that typically connect using a wireless communications medium such as cell phones, smartphones, Tablets or PDAs. Similarly, the client device may be any device that is capable of connecting using a wired or wireless communication medium such as a PDA, POCKET PC, wearable computer, and any other device that is equipped to communicate over a wired and/or wireless communication medium.

The user's device may include a browser application that is configured to receive and to send web pages, web-based messages, and the like. The browser application may be configured to receive and display graphics, text, multimedia, and the like, employing virtually any web-based language, including Standard Generalized Markup Language (SMGL), HyperText Markup Language (HTML), and so forth.

The system is designed to have two ends. One is the front end which is designed as the front panel with GUI for taking the user's input and displaying various types of outputs and the second one is the back end which is the cloud-based server. All information entered is stored, processed and evaluated in the database server according to the given format. The same database allows successful evaluation and withdrawal of information.

The system as per its preferred embodiments presents an interface allowing the user to sign up at the designated platform. The signup process is simple and provides a form interface that asks for basic information about the user to complete the registration process. The user can be asked for first and last name, role, user name, and password. Once the user provides the necessary information and selects the role, he/she can register by pressing the button. The user can be a HCSP or ITMS. Once the user in this case a ITMS signups up, he/she can register by fulfilling the necessary required information. If the user is a HCSP then the signup and search functions as per their required job requirement for the right resource for them. The ITMS can search for jobs and can apply. To apply for a job, ITMS needs to provide an available start date, expected end date, and other personal details including but not limited to home address, work address, CV, Medical license, and other Certificates. Once the ITMS application is submitted successfully, it goes to the review stage. If further information is needed, the user is notified. Else if no other action is needed from the applicant, the information is passed to HR which can approve and finalize the rest of the details.

In accordance with the method, a profile database is provided, which stores information about the ITMS and provides a real-time availability of the health care staff resources. The information stored about the health care staff resources includes availability, services being offered, geographic service area, accreditation, experience, etc . . . .

Also in accordance with the method, a service request message is received from a HCSP via a first communication device. The service request message includes a description of a requested service, a date and time period for the performance of the requested service and a location where the requested service is to be performed. The preferences of the ITMS are retrieved from the profile database. A job specification is created from the service request message and the retrieved preferences of the ITMS. The job specification includes the ITMS fee rate, experience, and availability. Information about resources is retrieved from the profile database. The job specification is compared with the retrieved information about resources to identify resources that meet predetermined minimum requirements of the job specification.

Rules are applied to the job specification and the retrieved information for the identified resources to sort the identified resources into categories comprising exact matches. Real-time triggers will notify ITMS(s) of available job shifts for which they qualify. On a first-come-first-serve basis, ITMS(s) can select and commit to future work shifts. After selecting, the ITMS will also receive a notification-of-acceptance message.

Besides the ITMS, the HCSP will also receive a message that the available work shift has been accepted and confirmed.

Once a ITMS is approved, multiple features like clock in and clock out, time cards to show hours of working, and shift management features are also available. The shift management feature finds new locations based on the healthcare staffs current location. Users can click on available dates for opened/available locations and can book their slots. If health care staff isn't able to join a particular shift, he/she can cancel the shift which triggers notifications to other ITMS listed on the same locations.

As per further embodiments, the revenue stream is based on a commission fee from the ITMS. The total value of each shift includes the doctor-in-training payment, fees, taxes, and company commission. Furthermore, a second revenue stream is the subscription fee from the HCSP to use the automated features of the platform to maintain constant coverage for shifts.

It is appreciated that additional advantages, modifications and equivalent embodiments will be apparent to those skilled in the art. Therefore, the invention, in its broader aspects, is not limited to the specific details shown and described herein. Accordingly, various modifications may be made without departing from the spirit or scope of this invention as defined by the appended claims and their equivalents.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The invention claimed is:

1. A method for arranging and overseeing the provision of health care services between ITMS and HCSP in a peer-to-peer services marketplace computer system, the method comprising:
a profile database storing information about the ITMS and the HCSP, the information stored about the ITMS including availability and services being offered.
the information stored about the HCSP will include details about current open shift(s) and add any specific details related to the work shift.
the ITMS will receive a communication that includes a description of a service, a date and time period for performance of the requested service and a location where the requested service is to be performed.
the HCSP will receive a message that the job shift(s) have been accepted by the ITMS and confirmed.
